(19) United States
(12) Patent Application Publication
(10) Pub. No.: US 2009/0151737 A1
(43) Pub. Date: Jun. 18, 2009

(54) FOCUSED ATTENTION AND PAIN REDUCTION

(76) Inventor: Amy Lynn BAXTER, Atlanta, GA (US)

Correspondence Address: LEWINSKI LAW GROUP LLC, 4508 EXMOOR DR., MARIETTA, GA, 30067, US

(21) Appl. No.: 12/371,206

(22) Filed: Feb. 13, 2009

Related U.S. Application Data
(63) Continuation-in-part of application No. 11/867,630, filed on Oct. 4, 2007.
Application No. 11/867,630 is a continuation-in-part of application No. 11/538,718, filed on Oct. 4, 2006.

(51) Int. Cl. A61B 17/00; A61H 1/00; A61F 7/00; G09F 1/02; G03B 25/02

(52) U.S. Cl. 128/898; 601/46; 607/96; 40/124.01; 40/454

(57) ABSTRACT

A focus card evokes a neurological response in a user to dampen the user's response to a negative stimulus. A focus card has a first face comprising visual graphics such as lenticular graphics, and a second face comprising indicia pertaining to the visual graphics. Indicia are configured for a target population, and can include novel questions, tasks or directions. A parent or other untrained individual can direct a patient as indicated by the indicia. A single focus card can have indicia for several target populations. A focus card can be used in combination with an analgesia means to further reduce a patient's pain response. By focusing the patient's attention with a focus card, behavior of the patient's anterior cingulate gyrus can be modified to reduce the patient's response to a pain-causing stimulus. It can also be used to reduce agitation, anxiety or distress outside of a medical environment.

This application is a continuation-in-part of copending U.S. patent application Ser. No. 11/867,630 filed on Oct. 4, 2007 entitled APPARATUS AND METHOD FOR THE REDUCTION OF PAIN USING VIBRATION, COLD AND DISTRACTIVE ELEMENTS. U.S. application Ser. No. 11/867,630 is a continuation-in-part of copending U.S. patent application Ser. No. 11/538,718 filed on Oct. 4, 2006 entitled APPARATUS AND METHOD FOR THE REDUCTION OF PAIN ASSOCIATED WITH NEEDLE STICKS. The disclosures of both of the applications identified above are incorporated herein in their entirety by reference.

### FIELD OF INVENTION

This invention relates generally to means for reducing pain perception in a patient. More particularly, the present invention relates to systems and apparatus for reducing pain by focusing a user's attention.

### BACKGROUND OF INVENTION

Medical procedures are often painful for patients. Needle pricks and injections at birth commence a long series of painful procedures, such as vaccinations, blood tests, and the like, that are conducted over a patient's lifetime to safeguard and improve his health. Should a patient become injured or ill, the number and type of painful procedures to which he must undergo increases. While patients are often sedated for surgical and invasive procedures, other procedures such as hypodermic needle administrations, lumbar punctures, debridement procedures, wound dressing changes, foreign object removal, cryogenic treatments, physical therapy, suturing procedures and the like, while painful, are typically conducted without the accompaniment of pain management techniques. The typical six-year-old child has received 36 intramuscular vaccinations; yet research has shown that only about 6% of pediatric practices offer pain control when administering these shots. Approximately 18 million venipunctures are performed annually without the accompaniment of any type of pain management procedure. In addition to short-tem discomfort, unmanaged pain can have long-term effects. Studies have shown that unmanaged pain during a procedure can cause a heightened pain response to a subsequent procedure.

Because they associate health care providers with pain, many patients, particularly pediatric patients, learn to fear and dread visits to a physician or other medical practitioner. In addition to the pain associated with the visit, children may be frightened and intimidated by unfamiliar medical staff, apparatus, and the sights, sounds, and smells intrinsic to a medical facility environment.

Appreciating this fear and trepidation, parents and care providers may delay or avoid visits to a doctor's office. Unfortunately, a delay in seeking medical attention for a current condition often leads to a worsening of the condition, which, when treated, ultimately leads to an increase in the pain and anxiety experienced by the patient. In addition to delaying attention for a current or chronic condition, fear of pain may discourage patients from seeking preventative care such as check-up exams, follow-up visits and vaccinations.

Studies have correlated painful childhood experiences with poor attitudes toward health care in adulthood. Procedures associated with hypodermic needles or other medical sharps are particularly problematic. Adults as well as children may dread needle sticks to the extent that they avoid annual flu shots, diagnostic blood tests, or other needle-related procedures. Such avoidance could not only prove deleterious to the individual's health, but could seriously compromise the health of those around him as well, particularly if the individual has a contagious disease. For example, needle-aversion may discourage or prevent people from requesting an HIV-test, potentially delaying life-saving treatment and unwittingly increasing exposure of the AIDS virus to others. Health care providers themselves are sensitive to the pain and fear associated with medical procedures. A survey of practitioners has shown that they are six times less likely to administer all vaccines during one visit if 3 or more vaccinations are due.

In addition to conditioning a fear response in young children, early painful experiences can also impede normal physiological development of pediatric patients. Over the course of research conducted with human infants and rat young, it has been observed that in response to experiencing pain at an early age, the manner by which the subjects perceive pain, and the development of the subjects' neurological connections can be altered. From a practical perspective, a fearful and anxious child may be more difficult to control, and thus require additional personnel to restrain him during the procedure. Enlisting the assistance of additional staff members not only slows office operations, but can also exacerbate the anxiety and distress of the patient.

In the past, various means have been used to decrease the pain associated with medical sharps. Topical analgesics have been employed, but they require lengthy application times and can cause vasoconstriction that decreases venipuncture success. In addition, the costs of analgesics can range from $12 USD to $150 USD per needlestick. Because the nerve fibers that transmit pain also transmit perceived cold and vibration, cold spray has been used at a needle site to decrease the pain associated with a hypodermic needle. Unfortunately, in addition to causing vasoconstriction, cold spray has been observed to actually increase distress in pediatric patients.

Vibration has been used to reduce the pain of dental injections, but vibrating needles have failed to prove effective in reducing pain in other dental procedures. While a hand-held vibrating massager has been successful in reducing injection pain in adults, it has not been tested conclusively in children. In addition, the cold spray and vibratory means described above have typically been applied at the intended injection site, which can often cause logistical difficulties for the medical practitioner and anxiety for the patient.

Various distraction methods have been employed to decrease pain sensation. For example, playing music has been found to be an effective method for lowering the pain associated with childbirth and cancer treatments, as well as the chronic pain endured by patients with chronic conditions. Visual distraction techniques have also been employed. For example, some physician's offices are decorated with murals, posters, photographs or other artwork to relax a patient. While such displays have been successful in reducing a patient's stress, and anxiety, their effectiveness in reducing pain during a medical procedure is less clear. Alternatively, some medical offices are equipped with video equipment for showing movies. However, video devices can be expensive to purchase and install. In many cases the video equipment is located in a waiting room, rather than a treatment room, as private treatment rooms may be too small to accommodate the equipment. Thus, while the patient may be able to watch a movie while he is waiting to be summoned, he may no be able to watch a movie while he is in the treatment room undergoing the procedure. When deployed in a clinical setting in which several patients are treated in a single room, the video program may not be visually accessible by all patients, depending on the line-of-sight from the patient to the display screen, and the glare from the screen. Furthermore, when a single video presentation is shown to several patients of various ages, some patients may be interested in the movie, while others are not.

Individual virtual reality systems have also proven successful in reducing a patient's perception of pain. For example, virtual reality goggles can provide an attractive alternative environment for a patient with which he can interact. However, virtual reality systems can prove to be expensive. In addition, goggles may not be properly sized for young children, such as pediatric patients aged four and under. Similarly, children two years of age and younger may become uncomfortable or agitated with goggles strapped to their heads. Alternatively, if the goggles are mounted on a separate support structure without attachment to the patient, the patient may turn away from the device rather than become engaged in the alternative environment. Furthermore, any equipment used in a clinical environment, particularly that of a pediatric practice, must be easily cleaned and sterilized to prevent transmission of disease. In addition the device must be sufficiently durable to withstand handling by children and contact with body fluids.

In some cases coaching by a companion or care provider has helped reduce a patient's anxiety or pain. However, in most instances the person serving as a coach must receive proper training prior to assisting a patient during a medical procedure. Unfortunately, training materials and classes are not readily available to the general public, so the number of individuals qualified to effectively reduce a patient's perception of pain is limited.

### SUMMARY OF THE INVENTION

The present invention provides apparatus, systems, and methods for reducing pain perception by focusing a user's attention on a positive stimulus. By directing and focusing the user's attention, practice of the invention can modify the user's brain activity to produce a neurological response that reduces the user's perception of pain. Specifically, by focusing the user's attention on a positive stimulus, the invention increases the user's neurological response to the positive stimulus, which in turn decreases the user's neurological response to a negative stimulus. When the neurological response to a pain-causing stimulus is dampened, the patient's perception of pain is decreased. Other analgesic means, such as thermal and/or vibratory analgesia, can also be employed to further dampen the brain response to a negative or hurtful stimulus.

A focus card is configured to focus the user's attention and stimulate a neurological response that dampens the user's response to a negative stimulus. A focus card provides an effective and economical means by which pain management can be individually tailored for a patient. In an example embodiment, a focus card comprises two opposing faces. A first face of the focus card has visual graphics covering a substantial portion thereof, and a second face has indicia pertaining to the visual graphics and configured for focusing a user's attention. The indicia are configured for a target population identified on the second face of the focus card. The target population identification and the indicia configured for the target population are visually correlated by a common attribute so that the proper indicia are referred to for a user in a particular target population. The visual graphics are configured to be appealing and interesting. In an exemplary embodiment, the visual graphics are multi-colored. In a further example embodiment the visual graphics comprise lenticular graphics.

The indicia on the second face of the card are configured to focus a user's attention. In an example embodiment, the indicia comprise tasks or questions that can be directed to a user, and require a user to study the visual graphics in order to perform the task or answer the question. In an exemplary embodiment, the indicia comprise unfamiliar questions that require observation and study of the visual graphics on the focus card, thereby focusing and maintaining a user's attention. A companion of the user can direct the user as indicated by the indicia. The indicia are configured to allow an individual to direct the user in order to focus the user's attention, so that a parent, friend, volunteer, or other individual can interact with the user in the practice of the invention without having to receive prior training regarding use of the focus card.

In an exemplary embodiment, the indicia comprise tasks configured for a target population. A target population can include members sharing a common characteristic, for example, users of a predetermined age or developmental state. Identification of the target population is shown on the focus card. For example, knowledge of the cognitive, neurological, and physiological development of a pediatric patient ranging in age from 4-6-years-old can be used to configure questions or tasks that can be directed to a user in that age group in order to focus his attention and dampen his pain response. Indicia can be arranged in a plurality of groups, with each group intended for a different target population. Identification of the target population and the indicia configured for the target population can be visually correlated by a common attribute.

In an exemplary embodiment, a plurality of focus cards are retained by a retention means configured to retain the focus cards in a manner that allows an unobstructed view of both opposing faces. In an example embodiment, the retention means is configured to allow a plurality of focus cards to be used in succession to retain the user's attention and interest, thereby stimulating a brain response that dampens the patient's pain perception. In an example embodiment, the retaining means is a ring that allows rapid flipping of the focus cards. Short transition times between cards helps to maintain the user's attention and thereby dampen his response to a negative stimulus.

In an example embodiment, a method for focusing a user's attention and dampening his response to a negative stimulus comprises displaying to a user a first face of a focus card, the first face comprising visual graphics over a substantial portion thereof, the focus card having a second opposing face comprising one or more indicia pertaining to said visual graphics and configured to focus the user's attention, the indicia configured for a target population identified on the second face, and directing the user as indicated by said indicia to focus the user's attention. In a further example, a method can include displaying the first face of the focus card and directing the user while a negative or pain-producing stimulus is applied to the user.

In addition to a focus card, a system can include an analgesic means. An example system can include a housing configured to accommodate an analgesic means and apply said analgesic means to a user's body, and a focus card having first and second opposing faces, said first face having visual graphics on a substantial portion thereof, and said second face having indicia pertaining to said visual graphics, said indicia configured to focus a user's attention on said visual graphics. The analgesic means can comprise a thermal or a vibratory means, or both. In a further embodiment, the housing comprises a receiving means for receiving and retaining said focus card, wherein the receiving means is configured to receive and retain the focus card so that the user can view the visual graphics on the first face while the housing is positioned on said patient's body. The focus card can be positioned to obstruct a patient's view of the medical site, further alleviating the patient's anxiety.

An example method can include displaying a focus card to a user, directing a user as indicated by the indicia, and applying an analgesia means to said user's body. For example, a housing can accommodate a thermal analgesic means and/or a vibratory analgesic means. The housing can be positioned at a location proximate to the site receiving medical attention so as to optimize the pain-reducing effects of the thermal and/or vibratory analgesic means. In a preferred embodiment, the thermal and/or vibratory analgesic means are activated prior to and during the medical procedure to maximize the analgesic effects for the patient. Thus the present invention can utilize a combination of analgesic means in addition to attention focusing means to reduce a patient's pain and anxiety before and during a medical procedure.

Thus a focus card is a safe, effective, economical means for tailoring pain management for a patient. In an exemplary embodiment a focus card is sized for easy observation as well as easy manipulation. Thus, a focus card can be held and oriented as necessary to facilitate user observation. In addition, because the indicia are configured for a target population, indicia that maintain the attention of, and evoke the desired neurological response in each individual can be selected.

### BRIEF DESCRIPTION OF THE DRAWINGS

The patent or application file contains at least one drawing executed in color. Copies of this patent or patent application with color drawing(s) will be provided by the Office upon request and payment of the necessary fee.

FIG. 1 is a perspective view of an example embodiment.

FIG. 2A depicts example embodiments of a focus card.

FIG. 2B depicts the examples of FIG. 2A in color.

FIG. 3 depicts an example embodiment wherein a plurality of focus cards are retained by a retention means.

FIG. 4 depicts a further example of a focus card.

FIG. 5 shows a flow diagram of an example method.

FIG. 6 depicts a perspective view of an example embodiment.

FIG. 7 depicts an example of a housing.

FIG. 8 depicts a further example housing.

FIG. 9 depicts a further example housing.

FIG. 10 shows a flow diagram of an example method.

### DESCRIPTION OF EXAMPLE EMBODIMENTS

The present invention provides systems, apparatus and methods for reducing pain and anxiety in a patient. Exemplary embodiments are described below and discussed with reference to the appended drawings in which like reference numerals refer to like elements throughout the several views. While disclosed in order to fully teach the practice of the present invention, the embodiments discussed herein are merely examples of an invention that can be variously practiced, and, as such, are not to be considered limitations. Drawings are provided to complement the text and further enhance understanding of the invention; however the drawings are not to scale, and in some cases features may be omitted in order to emphasize novel aspects. Flow diagrams depicting methods are included, with discrete blocks denoting actions performed during a process. However, no chronological order limitation is implied by the flow diagrams, and the various discrete actions can be combined without straying from the scope of the invention.

“Hypodermic needles” as used herein includes, but is not limited to, all different types of needles, pins, probes, sharps and the like that can be used to inject, remove, or apply fluids, medications and the like to a patient, as well as to conduct tests such as but not limited to allergy tests to a patient.

“Thermal analgesia” as used herein includes, but is not limited to, the use or application of cold or reduced temperature (or removal of heat) to a patient to induce an analgesic effect.

“Vibratory” or “vibrational” analgesia as used herein includes, but is not limited to, the use or application of vibration to a patient to induce an analgesic effect.

“Vibrational and thermal analgesia” as used herein includes, but is not limited to the use or application of both cold or reduced temperature (or the removal of heat) concurrently, substantially concurrently, or sequentially with the use or application of vibration to a patient to include an analgesic effect.

“Indicia” as used herein includes, but is not limited to one or more printed messages, instructions, indications, questions, tasks, information, directives, notations and the like conveyed by text and/or symbols.

“Stimulus” as used herein includes, but is not limited to something causing or regarded as causing a response; an agent, action, or condition that elicits or accelerates a physiological or psychological activity or response.

“Positive” as used herein includes, but is not limited to non-painful, non-hurtful, pleasant, beneficial, desired, not undesired.

“Negative stimulus” as used herein includes, but is not limited to undesired, hurtful, painful, capable of causing pain or injury, nociceptive, unpleasant.

“Graphics” as used herein includes, but is not limited to concrete or abstract photographs, drawings, images, patterns, objects, characters, shapes, colors, designs, pictorials and the like.

“Lenticular” as used herein includes, but is not limited to two-ordimensional graphics that can show special effects such as motion or action sequencing, flipping from one image to another, three-dimensional graphics , or graphics having the appearance of depth.

“Anterior Cingulate Gyrus” includes the frontal portion of the gyrus which is in the medial portion of the brain and partially wraps around the corpus callosum. It receives inputs from the thalamus and the neocortex as well as from somatosensory areas of the cerebral cortex.

“Anterior Cingulate Cortex” includes the frontal portion of the cingulate cortex, which is formed around the corpus callosum, which is the fibrous bundle that relays neural signals between the right and left hemispheres of the brain.

“Cerebral cortex” includes that portion of the brain covering the outer portion of the cerebrum and cerebellum, and is divided into left and right hemispheres. The cerebral cortex is responsible for processing information received from the sensory systems. The parietal lobe is particularly involved in receiving sensory information, while the frontal lobe of the cerebral cortex is particularly involved in problem solving.

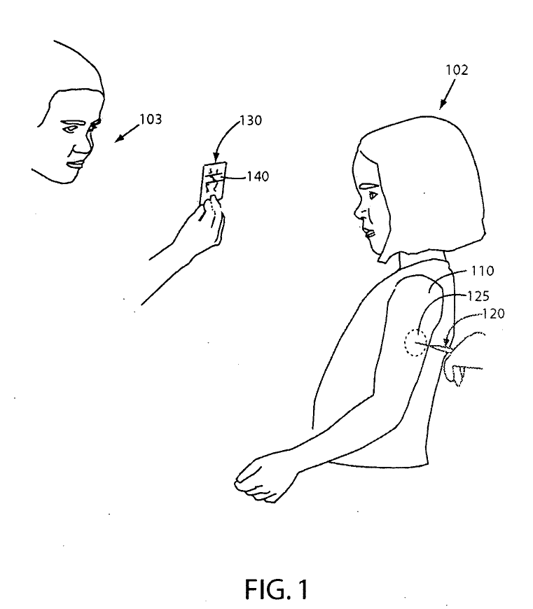

FIG. 1 shows an example embodiment 100 of the present invention. A patient 102 is to receive an injection with a hypodermic needle 120 at a medical site 125 on his arm 110. The patient's gaze is directed to a focus card 130 having visual graphics 140. By engaging and focusing the patient's attention, the embodiment 100 can reduce the patient's pain response to the hypodermic injection without the use of expensive pharmaceutical drugs that may have unintended side effects or invasive techniques that may be uncomfortable. An individual 103 can cooperate with the user 102 in the practice of the invention. The individual 103 can be a person other than a medical practitioner, and need have no prior knowledge or training regarding the implementation or operation of the invention.

Advances in the knowledge and understanding of psychological and neurological factors contributing to the sensation of pain have led to an increased interest in the use of non-invasive methods of reducing a patient's pain perception. Studies show that when a subject focuses his attention on a particular stimulus, electrical activity in the brain is higher in response to the attended-to stimulus, than in response to other stimuli to which the patient is not giving attention. (See van der Veen F M, van der Molen M W, Jennigs J R. Selective attention and response inhibition alter phase-dependent cardiac slowing. Psychophysiology 2001; 38(6):896-902, which is incorporated herein in its entirety be reference). These observations are consistent with results predicted by the Gate Control Theory (GCT) of pain (See Melzack R, Wall P D. Pain mechanisms: a new theory. Science 1965; 150(699):971-9, which is incorporated in its entirety by reference). The GCT describes the pain perception process as follows. Peripheral nerves become excited in response to cell damage caused by a harmful stimulus. Via the spinal cord system, the excited peripheral nerves transmit ascending nerve impulses to the cerebral cortex of the brain where pain is perceived. The conduction of nerve impulses toward the brain is controlled by the substantia gelatinosa which functions as a gate to the cerebral cortex. According to the GCT, both external forces, such as rubbing the person's skin, and internal forces, such as a person's fear and anxiety, can affect operation of the substantia gelatinosa. Thus, by modifying the substantia gelatinosa behavior, a patient's perception of pain can be altered. The amount of nerve input received at the spinal cord system can also influence substantia gelatinosa performance. When there are more nerve impulses traveling to the brain from a non-painful (positive) stimulus than from a pain-causing (negative) stimulus, the substantia gelatinosa gate can block some or all of the pain-producing impulses from reaching the cerebral cortex.

In addition, the noxious inhibitory controls theory (NICT) of pain suggests that descending impulses sent in response to a distracting stimulus can interfere with ascending impulses carrying a pain signal, reducing the number of ascending impulses that reach the brain, and thereby diminish pain perception. Accordingly, generation of sufficient impulse throughput in response to a non-painful stimulus can reduce the pain perceived by a patient.

The anterior cingulate gyrus (ACG) portion of the brain also has a role in a user's perception of pain. Research has shown that a pain-producing stimulus can induce potentials across the ACG of a human brain; leading to the conclusion that nociceptive stimuli trigger metabolic activation of the ACG. Thus, in an effort to reduce a patient's pain perception, an attempt can be made to modulate the pain and anxiety mediated by the ACG by stimulating cognitive activity by a patient.

The focus card 130 is configured to capture a user's attention, producing a neurological response that reduces the user's sensation of pain. The focus card 200 can be considered a positive stimulus, in that the focus card is configured to evoke a response that is not characterized by pain or agitation. In an exemplary embodiment a focus card is configured to induce a neurological response that dampens the user's response to a pain-causing or negative stimulus. In an example embodiment, a focus card is configured to evoke a neurological response in which ascending and descending impulses transmitted along the user's spinal column system interfere with ascending impulses generated in response to a negative stimulus. In a further embodiment, a focus card is configured to evoke a neurological response in which impulses generated in response to a negative stimulus are prevented from passing through the substantia gelatinosa. A focus card can also be configured to activate and modify behavior of the ACG which mediates pain so as to reduce the pain perceived by a patient.

For example, referring to FIG. 1, injection with the hypodermic needle 120 can be considered a negative stimulus. It is an undesired stimulus that can excite the peripheral nerves, prompting them to generate impulses conducted along the spinal cord system that result in a sensation of pain. But when the user's attention is focused on the focus card, the pain sensed from the injection is diminished because fewer of the impulses generated in response to the injection are passed through the substantia gelatinosa to the cerebral cortex. In addition, the anterior cingulate gyrus (ACG) portion of the brain plays a role in pain perception by a patient.

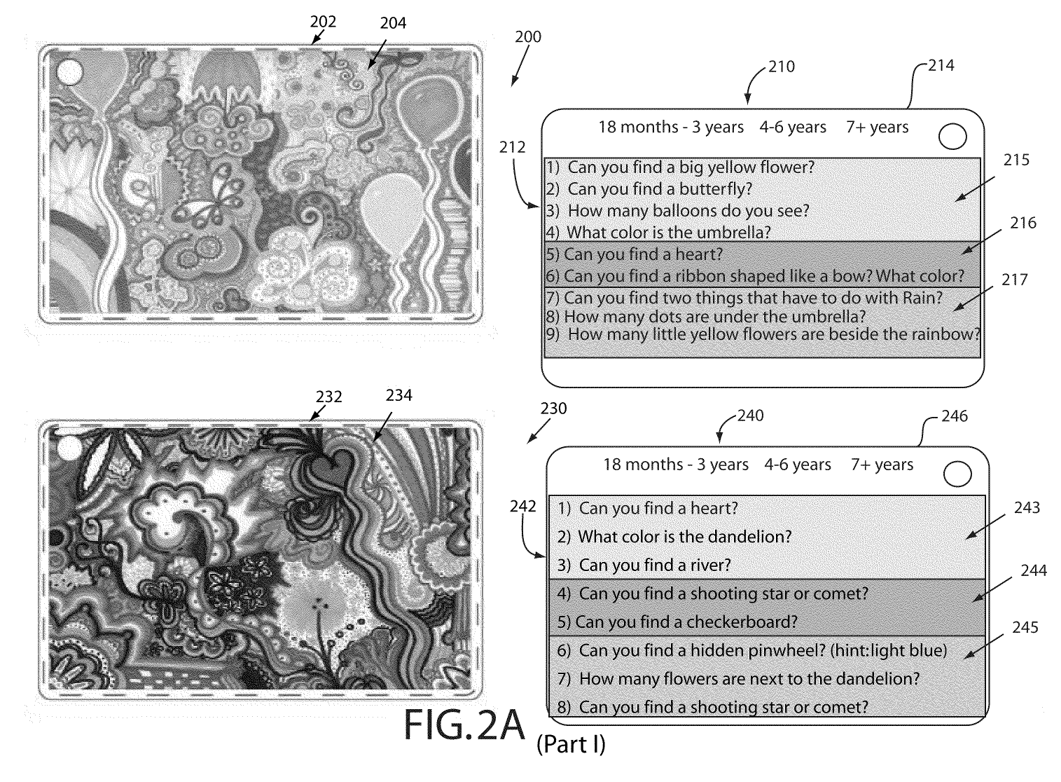

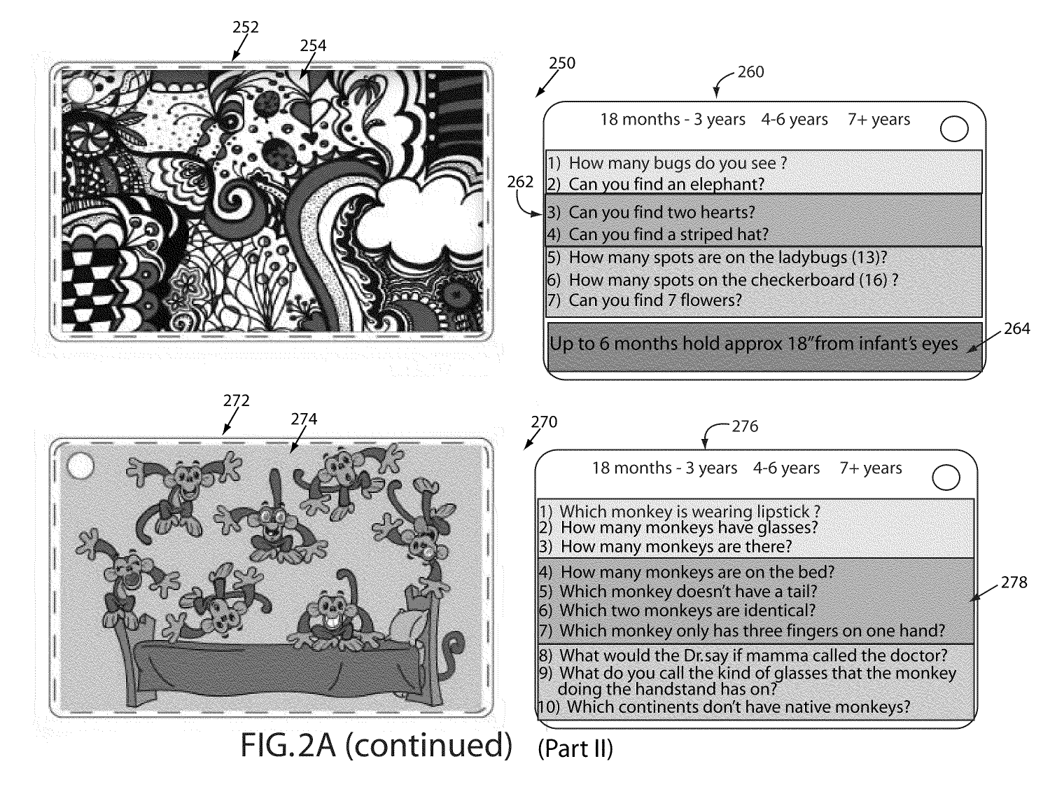

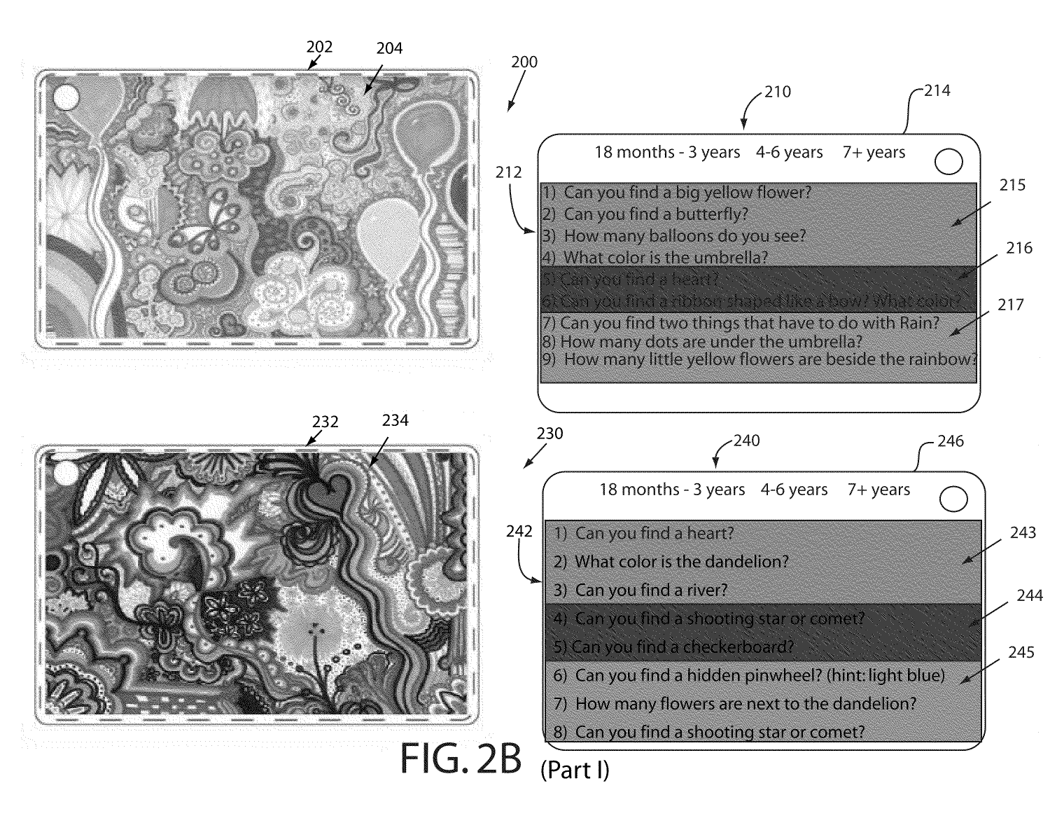

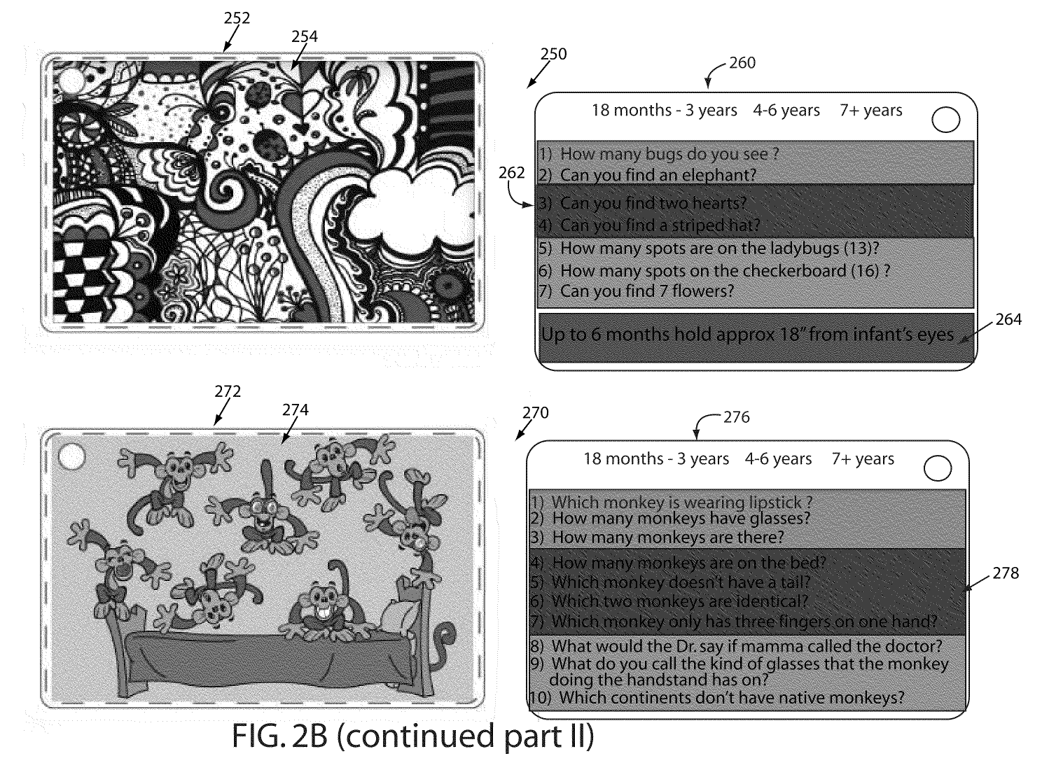

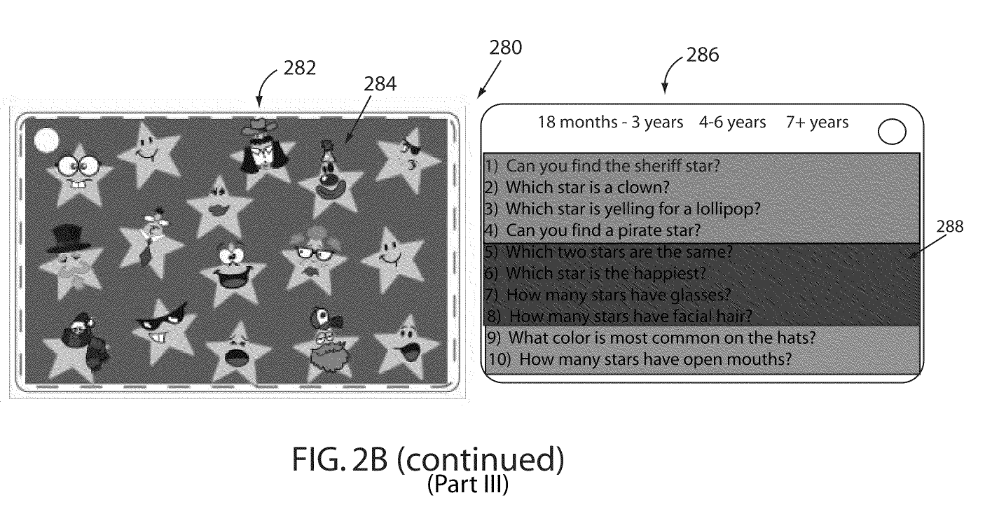

FIGS. 2A,B show examples of first and second opposing faces of example focus cards. FIG. 2B is a color depiction of the examples of FIG. 2A. A first face 202 of a focus card 200 contains visual graphics 204. In an exemplary embodiment, visual graphics cover a substantial portion of a face of a focus card. As shown in FIGS. 2A,B except for the peripheral margin 206, the visual graphics 204 cover the entire face 202. The visual graphics 204 can be depicted in colors and/or gray-scale, however multi-colored graphics are preferable to monochromatic graphics. Preferably the graphics are appealing and interesting to a viewer. Particular color selections and combinations can be employed to provide a desired level of contrast to improve patient visibility, interest, and focus, as well as to stimulate particular areas of the brain. In an exemplary embodiment, the visual graphics 204 comprise unique novel images, designs, patterns, and the like that are unfamiliar to the user. In an example embodiment the visual graphics 204 comprise lenticular graphics that appear as three-dimensional and/or non-stationary images. The visual graphics 204 can also include holographic images and the like.

A second face 210 of the focus card 200 includes indicia 212 pertaining to the visual graphics 204 on the first face 202 of the focus card 200. The indicia 212 can include, but not be limited to, instructions for using the focus card 200, as well as questions, tasks, or other activities pertaining to the visual graphics 204 that can be directed to a user. Preferably, the indicia 212 comprise one or more tasks or questions configured to require a user to focus on the visual graphics 204 in order to perform the particular task or answer the particular question. In an exemplary embodiment, the questions or tasks concern subject matter that is unrelated to a user's prior knowledge, exposure, experience, or previously memorized information. While a user's memory is associated with the temporal lobe and hippocampus portion of the brain, the indicia 212 can be configured to engage or activate the frontal and/or parietal lobe portions of the brain. In addition, the indicia 212 can be configured to modify behavior of the (ACG).

A focus card triggers a neurological response that is different from that triggered by prior art games or materials that prompt a player to find an object in a picture. Prior art picture books or puzzles typically ask a user to find a particular object, such as a well-known character or a referenced object, i.e. an object previously shown to the user then subsequently referred to. However, because the character or object is shown to the reader prior to asking the reader to find the character, the character is not considered “novel” subject matter. Thus, a search for the referenced object is merely a visual comparison of the object and the picture in which it is hidden. Furthermore, prior art puzzles are typically designed to allow a child to be self-directed. In most cases, a child needs little or no instruction from an adult. While a pre-reader may receive general directions prior to searching for the referenced objects, once a child begins to look for an object he can look for subsequent referenced items without new directions since the directions are essentially the same throughout and merely require the user to find a particular referenced object.

A focus card does not provide a reference object and then prompt a user to locate the reference object. Instead, a focus card prompts a user to find a “novel” object, which is not previously provided or previously referenced. The novelty of a stimulus has been shown to increase electrical activity in the ACG. Furthermore, the increased ACG activity in response to a novel stimulus correlates with a decreased response to a painful event or stimulus. In addition, each indicia or instruction of a focus card is different from the remaining indicia, and provides an unexpected search item previously unknown to the patient. Thus the user does not merely receive a general instruction, but is given a plurality of novel instructions, each pertaining to a novel object or aspect of the visual graphics previously unconsidered by a user. In an exemplary embodiment, an individual such as a parent directs indicia to a user. Direction by an authoritative questioner further enhances a patient's pain relief by diverting the user and interfering with a previous train of thought, effects that cannot be obtained by a static self-directed search.

In an exemplary embodiment, the indicia 212 comprise tasks configured for a predetermined target population. A target population can be defined by chronological age, developmental state, gender, nationality, or other characteristic or parameter. For example, knowledge of the cognitive, neurological, and physiological development of a pediatric patient ranging in age from 4-6 years old can be used to configure indicia for a user in that age group to focus his attention and thereby dampen his pain response. Questions that are too easy for a user may fail to adequately engage the user's attention to achieve the desired neurological response. Likewise, questions, tasks, or directions that are too difficult for a user may cause the user to lose interest in a focus card, and let his attention wander rather than remain focused on a focus card.

The indicia 212 can be configured for the target population based on an understanding of the physiological and neurological development of the target population, so as to maintain a user's focus and attention. Because the indicia 212 are configured in this manner, an individual cooperating with the use in the practice of the invention need not have prior training in order to maintain the user's attention when using the focus card to reduce pain. The use of indicia configured for the target population relieves the accompanying individual of the responsibility of generating his own questions for the user, which may be too simple or too difficult to maintain the user's attention and generate the desired neurological response. When the user is no longer attentive, the nerve impulse throughput necessary to sufficiently block or interfere with the nerve impulses from the painful stimulus is no longer generated, and the pain-reducing effect of the stimulus can be reduced or lost entirely.

The indicia 212 are configured to allow an untrained individual to use the focus card to focus the user's attention, so that a parent, friend, companion, volunteer, or other individual can interact with the user in the practice of the invention without having prior specialized knowledge or training regarding use of the focus card 200. When a person other than a medical practitioner, for example a family member or other familiar and trusted individual, interacts with a patient in the user of a focus card, the individual can provide comfort than can further reduce the pain and anxiety of the patient. In addition, less medical staff time is required to conduct the procedure, making the procedure less costly in time and resources.

The indicia 212 can include items directed to different target populations so that a single focus card can be used effectively with users belonging to various target population groups. The indicia 212 can be arranged and grouped on the focus card 200 face 210 according to the target population for which they are intended. An identification or description of the one or more target populations can be included on the face 210. In an example embodiment, the identification or description of the target population is shown within a header portion 214 on the face 210, to be readily apparent. In an example embodiment, the identification of the target population is visually correlated with the corresponding indicia so that a user can readily identify appropriate focus cards and indicia for a particular patient. In an example embodiment, the identification and corresponding indicia share a common attribute or characteristic. For example, both can be associated with a common color or a common font type.

Referring to FIG. 2A, the indicia 214 are arranged in three groups: group 215, 216, and 217. The first group 215 includes indicia numbered 1-4 that are configured for patients aged eighteen to thirty-six months. To indicate that the indicia of group 215 are configured for users 18-36 months of age, the particular age range can be denoted or identified on the face 210 in the header portion 214, for example “18 months-3 years” can be printed in the header portion 214. The identification itself can be characterized by a predetermined attribute, such as, but not limited to a particular, color, font type, font color, font size, color pattern, background color behind text, associated color block, and the like. The predetermined attribute can also be associated with or used to characterize the indicia configured for that particular target population. In this way the identification of a first target population can be visually correlated with the indicia configured for the first target population. For example, the identification of the age range “18 months-3 years” can be denoted in green text, and indicia configured for users in the 18-month to 3-year age range can be denoted in green text, so that the identified target population is associated with the same text color as the indicia configured for that target population. Common color association need not be limited to text color. For example, the indicia configured for the 18-36-month age group can be associated with a green background, a green highlight, a green enumeration scheme, etc. as will occur to those in the art.

A second target population can be associated with a second attribute, For example, identification of an age group ranging from 4-6 years can be denoted in the header 214, with a blue color rather than a green color. The identification can be printed in blue text, or be surrounded with a blue color block, blue highlight, etc. The indicia of group 216 can also be associated with blue, for example with blue text, a blue color block, blue highlighting, etc. that associates them with the identified target population. Finally, a third group 217 of indicia can be configured for users aged 7 years or older. Identification of this age group can be shown in the header 214 and associated with a third color, for example yellow. Group 217 indicia can then be associated with the color yellow to indicate that they are configured for users of this particular target population, specifically users aged 7 years or older. The colors used to code or group the tasks can vary, but should allow a user to easily read the tasks, and be sufficiently contrasting to allow multiple groups to be readily distinguished. As mentioned previously, the attribute is not limited to color, but can include any other type of distinguishing characteristic. As an alternative to using color to identify questions as targeted to a particular population, font type, degree of boldness, enumeration schemes, or other methods of denotation and correlation can be employed and will occur to those skilled in the art.

Focus card 230 is another example of a focus card. Focus card 230 has a first face 232 with visual graphics 234 covering a substantial portion thereof, and a peripheral margin 236. The focus card 230 has a second opposing face 240 having indicia 242 and a heading portion 246 in which target populations are identified: “ages 18 months to 3 years”, ages “4-6 years”, and ages “7+ years”. Indicia 242 comprise groups 243, 244, 245 each configured for on of the identified target populations and visually correlated with the identification of the target population.

In a further embodiment, an identification of a first target population and the indicia configured for the first target population can be visually correlated by a first type of attribute, and an identification of a second target population and the indicia configured for that second target population can be visually correlated by a second type of attribute. For example, identification of a target population of users aged 18-36 months and the indicia configured for users of that age can be associated with a common color. Identification of a target population of users 4-7 years old and the indicia configured for users of that age range can be associated with a common font type.

By including the identification of the target population in a heading portion, the identification is easily referred to, facilitating finding a focus card and identifying appropriate indicia for a particular user. In addition, by consolidating the identifications, more room can be allocated for indicia on a focus card. However, it is contemplated that target population identifications can be provided elsewhere on a focus card as well, for example, appearing adjacent to a particular indicia.

While referred to as a “header”, header portion 214 need not be disposed at a top or upper region of the face 210, but can be disposed in a central or lower region. It is contemplated that the indicia 212 can include instructions for using a focus card with a particular target population. For example, focus card 250 has a first face 252 having visual graphics 254, and a second face 260 having indicia 262 comprising portion 264 which provides an instruction for using the focus card 250 with an infant aged up to 6 months.

FIGS. 2A,B also depict example focus card 270 having a first face 272 with visual graphics 274, and a second face 276 having indicia 278. A further example focus card 280 has a first face 282 with visual graphics 284 and a second face 286 having indicia 288.

A focus card can be made from any substance or material having sufficient rigidity to provide a substrate for visual graphics on a first face and indicia pertaining to the visual graphics on a second face. Examples of materials that can be used include, but are not limited to, paper, cardboard, cardstock, plastic, wood, and other natural and synthetic materials. A focus card can be laminated to resist soiling, increase durability, and facilitate cleaning of the cards, a particularly beneficial feature when used in a clinical environment. In a preferred embodiment, a focus card is made of plastic to be easily cleanable and thereby prevent transmission of germs and diseases. In addition, unlike objects composed of paper or cloth, a plastic focus card can withstand contact with body fluids.

A focus card can be any size so long as visual graphics on a first face can be visible to a user positioned proximate to an individual holding the focus card, and indicia on a second opposing face are legible to the individual cooperating with the user. It is preferable that a focus card be small enough for easy handling and manipulation, but large enough for easy observation by a user

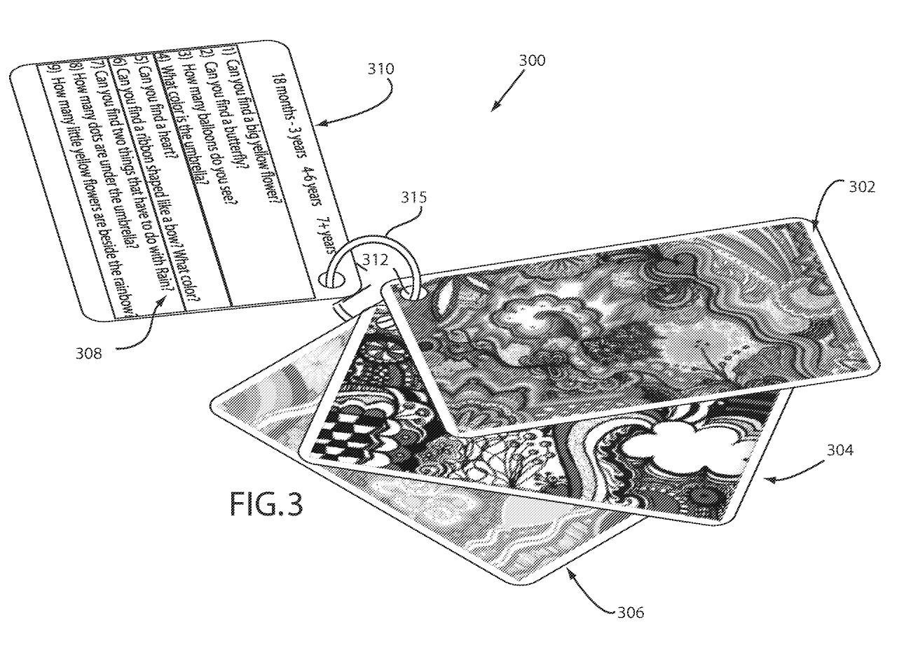

Referring to FIG. 3, in an example embodiment 300, one or more focus cards 302-310 can be retained on a retention means, such as a self-fastening device, or device coupled with a fastening or clasping means, or other type of retention device. For example, a ring 315 can be used to retain one or more focus cards. The focus cards 302-310 are configured for retention by a retention means. An aperture 312 in the corner of a focus card 302 facilitates removing and replacing the focus card 302 on the ring 315, and also facilitates rapid transitions between focus cards. When a patient finishes using a first focus card 302, a second focus card 304 can be quickly shown to the user in order to maintain the user's attention and desired neurological response. It is preferable that a retention means be configured to allow a second focus card to be displayed in less than 60 seconds to retain a user's attention, and more particularly in less than 5 seconds. A retention means can also comprise a housing configured to retain a focus card. Such a housing can be a stand-alone housing either held by an individual directing the user, or positioned on a surface, or can be a housing configured for attachment to the user.

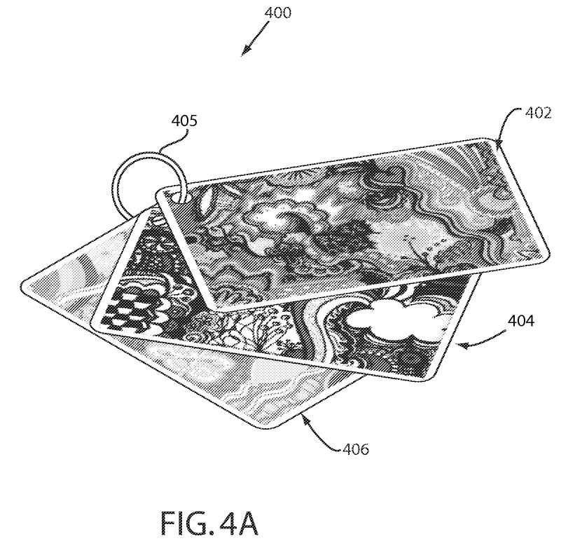

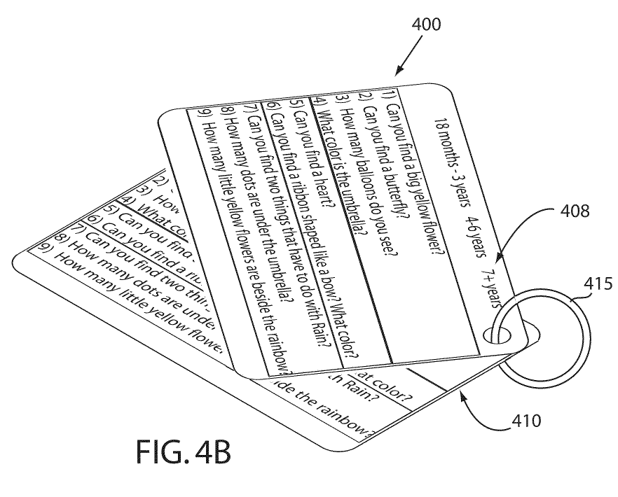

In yet a further embodiment, a focus card can have visual graphics on a first face, and be paired with a separate corresponding companion card having indicia pertaining to the visual graphics. FIG. 4A shows focus cards 402, 404, and 406 having visual graphics on a first face. When this embodiment is practiced, a focus card can be held by the patient, or positioned with a support means or housing proximate to the patient to facilitate his observation of the visual graphics. FIG. 4B shows example companion cards 408 and 410 that correspond to focus cards 402 and 404. The corresponding companion card can be held by or positioned at a separate location proximate to the individual cooperating with the user in the practice of the invention. The companion individual can direct the patient as indicated by the indicia on the companion card. Pairs of focus and companion cards can be identified and labeled so that the individual can use the proper corresponding companion card for a particular focus card. The card faces of each pair member can be numbered to allow both the visual graphics card and the companion card to be printed on both faces, so that each face of a focus card corresponds to a single face of a companion card. FIG. 4B shows companion cards 408, 410 retained on a retaining means 415, shown here as a ring. Companion cards can be retained separately from the visual focus cards, or both visual and companion cards can be combined on a single retention means.

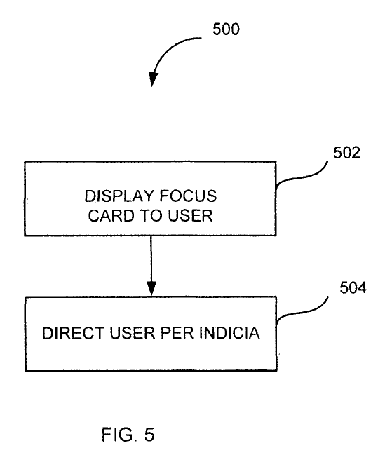

FIG. 5 shows an example method 500. At block 502, a focus card is displayed to a user. In an exemplary embodiment, the user is a patient about to undergo a medical procedure that involves a negative stimulus that typically causes the user to feel pain. As discussed previously, a medical procedure can include, but not be limited to, injections, debridement, wound treatments, oncology treatments, cryogenic treatments, and the like which would typically cause a patient to experience pain. Referring to FIG. 2, a first face 202 of focus card 200 having visual graphics 204 can be shown to the user. The focus card 200 can be positioned in close enough range for the user to see and observe the visual graphics 204.

At block 504, the user is directed as indicated by indicia pertaining to the visual graphics 204. In an example embodiment, an individual other than the patient directs the user as per the indicia. The individual can be a parent, caregiver, friend, medical practitioner, or other person, and need not have any prior training to cooperate with the user in the practice of the invention. The focus card itself performs as an instant trainer for the parent, providing the necessary information required to effectively practice the invention. In an exemplary embodiment, the individual refers to the indicia 212 on the opposing face 210 of the focus card 200. As shown in FIG. 2, the indicia 212 are arranged in three groups: 215, 216, and 217, each configured for a target population. The individual refers to the heading portion 214 to determine which group is applicable to the present user. For illustrative purposes, a user is 8 years old. An individual can see that the identification of the target population “7+ years” is visually correlated with group 217, as each is associated with the color yellow. The individual can refer to indicia in the third group, group 217, and direct the patient accordingly. For example, the user can be asked “Can you find two things that have to do with Rain?” Indicia of group 217 are configured to focus the user's attention on the focus card 200 and thereby generate a neurological response that dampens the patient's pain response to the medical procedure as explained above. When performed in a clinical context, it is preferable that method 500 be performed prior to subjecting a user to a medical procedure to initiate the desired neurological response, and be continued for a short period after the procedure has been concluded. A method can further include displaying a focus card and directing a patient as per indicia while a patient is undergoing a medical procedure. In a further example of a method, the focus card 402 is used, in which the corresponding indicia are presented on a companion card, rather than on an opposing face of a focus card.

It is also contemplated that the method 500 can be practiced outside the context of a medical environment to focus a user's attention to produce a desired neurological response that dampens an undesired response to a negative stimulus. For example, a focus card can be used with an autistic child to focus his attention in order to alleviate agitation or otherwise modify behavior. A focus card can be used to alleviate anxiety and/or distress in a user by inducing a neurological response in which nerve impulses in response to the focus card (positive stimulus) exceed or interfere with those resulting from a negative stimulus, whether the negative stimulus is a medical procedure or other event. For example, a focus card can be used in an educational environment to modify the behavior of a child who is anxious, upset, disruptive or is distracting other students. A variety of applications will occur to those skilled in the art.

It is noted that in an exemplary embodiment, an individual cooperates with a user to practice the invention. The interaction between the companion individual and the user further enhances the desired neurological response that dampens pain sensation. When a companion verbally directs the user, the user receives auditory signals that assist in focusing his attention and further contribute to the desired neurological response. Thus, the user is exposed to both auditory and visual stimuli that command his attention and generate a desired neurological response. Furthermore, the comfort a companion can provide can further suppress the pain response of a user by alleviating a user's anxiety. Because a focus card is configured to focus a user's attention to generate a desired neurological response, any individual can cooperate with a user in the practice of the invention without prior education and training, making the present invention an effective, economical, easy-to-use tool for reducing pain and anxiety by a user.

A focus card offers several advantages over prior art visual distraction methods and apparatus. Indicia are configured to maintain a user's focused attention so that the user's attention will not wander, as it may if the user is simply staring at artwork or photographs. Each indicia comprises a novel task, question or direction pertaining to a novel feature or aspect of visual graphics. In an exemplary embodiment, lenticular graphics feature three-dimensional and/or moving novel objects that further engage the interest and focus of a user. Indicia are configured for specific target populations, such as children 18 to 36-months old, and a single focus card can be used effectively with multiple target populations. Artwork or video programming displayed in a clinical environment may not be configured to engage the attention of a user of a particular target population, or may interest only a single target population. In addition, a focus card can be easily manipulated to position it in a location that is readily visually accessible to a user. Even if artwork or a video device is installed in a treatment room to distract a patient, a patient's position and orientation during a medical procedure may cause his line-of-sight to the artwork to be obstructed. Unlike the use of electronic video equipment to display a movie, which can be expensive and take up valuable space in a treatment room, a focus card is inexpensive and sized to be easily accessed and stored. A focus card is versatile and can be used by patients of varying ages and sizes, unlike virtual reality systems, particularly those that require goggles that are neither sized nor configured for small and/or very young patients.

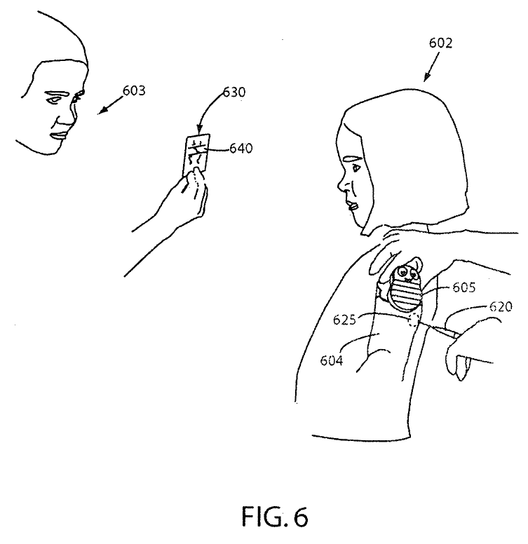

In a further embodiment, a system can include a focus card and a housing configured to accommodate an analgesic means and apply the analgesic means to a user's body. FIG. 6 shows an example system 600. A user 602 is to receive an injection in his arm 604 with a hypodermic needle 620 at a medical site 625. A companion 603 holds a focus card 630 that allows the user 602 to observe the visual graphics 640. A housing 605 configured for accommodating an analgesia means and applying said analgesia means to the body of user 602 is positioned on the user's arm 604 above the medical site 625. It has been found that placement of the housing 605 between a medical site and a patient's nerve plexia optimizes analgesia effects for the patient.

The housing 605 can have a hollow interior or interior sections for accommodating various operational elements. More specifically, the housing 605 can be a rigid hollow case having an interior volume or a flexible or pliant case having an interior volume. Such cases are known, as well as their materials and methods of construction. In an exemplary embodiment, a housing accommodates one or more analgesic means. For example, a thermal analgesic means and/or a vibratory analgesic means can be employed as disclosed in U.S. application Ser. No. 11/538,718 published as U.S. Patent Publication 2008/0086187 to Baxter, published Apr. 10, 2008, and U.S. application Ser. No. 11/867,630 published as U.S. Patent Publication 20080086063/A1 to Baxter and Calderon, published Apr. 10, 2008, both of which are incorporated in their entireties herein. The apparatus 600 can include a positioning means 615 configured to stabilize the housing 605 on the user's body. The positioning means 615 can include a patient contact means such as pads, legs, extensions and the like that provides an interface between the patient's body and the housing 605. The positioning means 615 can also include fastening means such as, but not limited to, hook and loop tape, snaps, buttons, buckles, hooks, etc.

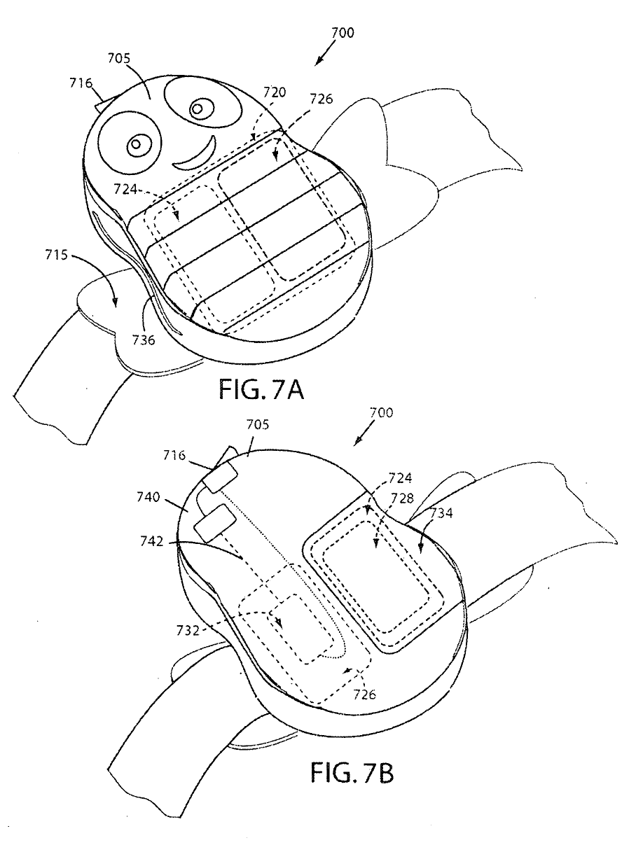

FIGS. 7A, 7B depict top and underside views respectively of an embodiment 700. The example device 700 has a housing 705 with an application area 720 adapted for applying one or more analgesic means. The application area 720 includes a thermal area 724, adapted for applying a thermal analgesia to a patient, and a vibratory area 726, adapted for applying a vibratory analgesia to a patient. Although depicted as discrete areas in FIG. 2, the thermal area 724 and the vibratory area 726 need not be physically distinct but instead can coextend and overlap. Although shown with both a thermal area 724 and vibratory area 726 for accommodating a thermal and a vibratory analgesia means, the invention can be practiced with only one or the other, with neither, or with an alternative analgesic means.

The housing 705 is a generally hollow structure sized to contain a thermal and/or vibratory analgesia means. Referring to FIG. 7B, the housing 705 includes a thermal source pocket 734 which can be a cavity, slot, opening, fold or other type of compartment into which a thermal source 728 can be placed. The thermal source pocket 734 is accessed on the side of the housing 705 via a mouth 736. The mouth 736 and thermal source pocket 734 can be disposed at various locations on the housing so long as the thermal source 728 contained therein can provide sufficient cooling effects to perform as an analgesia for the patient.

The thermal source 728 can be any conventional thermal source capable of storing and transferring cold, i.e. removing heat. Illustrative examples of suitable thermal sources include metal ingots, low freezing point (below 45° F.) liquids and gels, ceramics, polymers, other heat sinks, and even ice. Other thermal sources are known to those skilled in the art. The thermal source 728 is configured to transfer cold to the patient to the extent necessary to function as an analgesic. For example, when used in conjunction with a needle stick, providing a temperature below about 45 degrees F. and preferably between 380 and 450 F. is adequate for providing a suitable level of analgesia. The thermal source 728 is applied to the patient for a period of about 0 to 60 seconds prior to the needle stick to provide a suitable level of analgesia.

In its simplest form, the vibratory area 726 is an area on the device 700 that allows vibrations from a vibrational source 732 to contact the patient. Vibratory area 726 can be a pad or other area on the housing 705 in vibratory contact with the vibrational source 732, which is preferably contained within the main volume of the housing 705. The placement of the vibrational source 732 is variable so long as the vibration effects are sufficient to produce vibratory analgesia for the patient.

The vibrational source 732 can be any conventional vibrational source or means for producing vibrations. The vibrational source 732 can produce single, multiple or variable vibrational cycles. Examples of a vibrational source can include, but not be limited to elliptical flywheel motors, eccentric motors and the like as known in the art. The vibrational source 732 is configured to provide a sufficient level of vibration so as to provide an analgesic effect for the patient Preferably, the vibrational source 732 is able to provide vibrations of between about 300 vibrations per minute and about 9000 vibrations per minute, and more particularly between 3700 and 6000 vibrations per minute, and more preferably between 400 and 5800 vibrations per minute to provide a desired level of analgesia for a needle stick procedure. The vibrational source 732 is preferably applied to the patient prior to the needle stick for a time period sufficient to produce vibratory analgesia. The time period can be between 0 and 60 seconds, but is more preferably between 5 and 30 seconds, and more particularly between 5 and 10 seconds prior to a needle stick. Application of the vibrational source 732 can continue throughout the medical procedure.

The vibrational source 732 and power source 740 can be placed within the housing 705 during manufacture, or an ingress and egress means can be provided. The vibrational source 732 and power source 740 can be held within the housing 705 by friction, adhesives, fasteners or other types of securing means. In the example embodiment 700, wiring 742 electrically connects vibrational source 732 and the power source 740 to an on/off switch 716. The switch 716 used to turn the vibrational source 732 on and off can be a common switch as known in the art, and can be secured at any convenient position on the housing 705 so as to be accessible. Examples of switches include, but are not limited to, a toggle, lever, push-button, capacitance, switch, three-way switch, common potentiostat, or other switch.

In a further embodiment, a housing can be personalized by a patient. For example, a housing can be formed in a predetermined shape and size. A patient can customize the housing using various customizing elements such as, but not limited to, stickers, plastic or vinyl elements, static-cling elements, magnetic elements, labels, and the like. It is preferable that the customizing elements be removably attachable to the housing so that a single housing can be embellished in numerous ways simply by adding and removing a variety of customizing elements to the housing. Customizing elements can be formed in a plurality of shapes, colors and sizes, including but not limited to: eyeballs, noses, mouths, antennae, stripes, dots, ears, geometric shapes, legs, arms, articles of clothing, accessories, letters, numbers, sports-related items, etc. Pediatric patients can personalize the housing to make it appear like a character of their choice, or simply use the elements to decorate the housing to make it more attractive, interesting, or uniquely theirs.

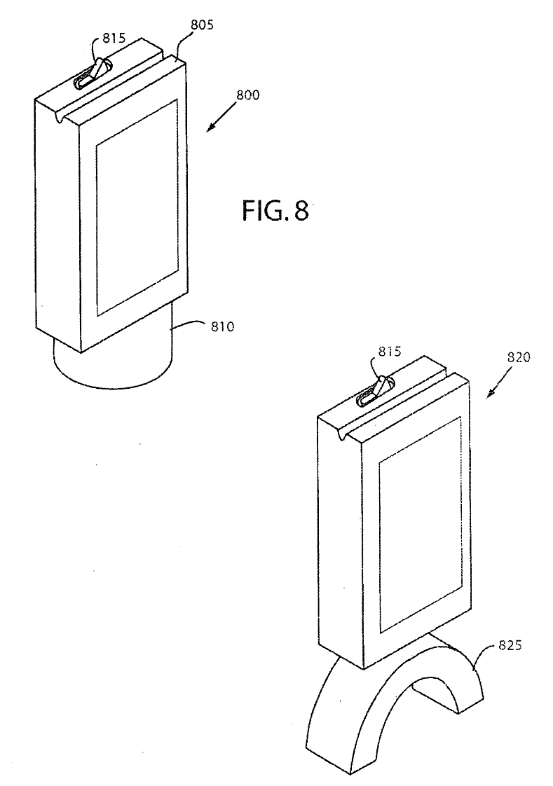

FIG. 8 shows a further embodiment 800 of the present invention having a generally vertical orientation. The device 800 includes a housing 805 and a positioning means in the form of a base 810. The base 810 performs as a skin contact member that can facilitate transmission of cold and vibration when the device 800 includes thermal and vibratory analgesic means. The base 810, depicted here as a pedestal, can be variously formed. For example, the base 810 can be in the form of a U-shaped member 825 of embodiment 820, a ring-shaped member, a set of one or more extensions, such as arms or legs, etc. When used in conjunction with an analgesic means such as a thermal source and/or a vibratory source, the analgesic means can be positioned within or around the base 810. The device 800 further includes a switch 815 for powering on and off an analgesic means, such as a vibratory means, accommodated by the device 800.

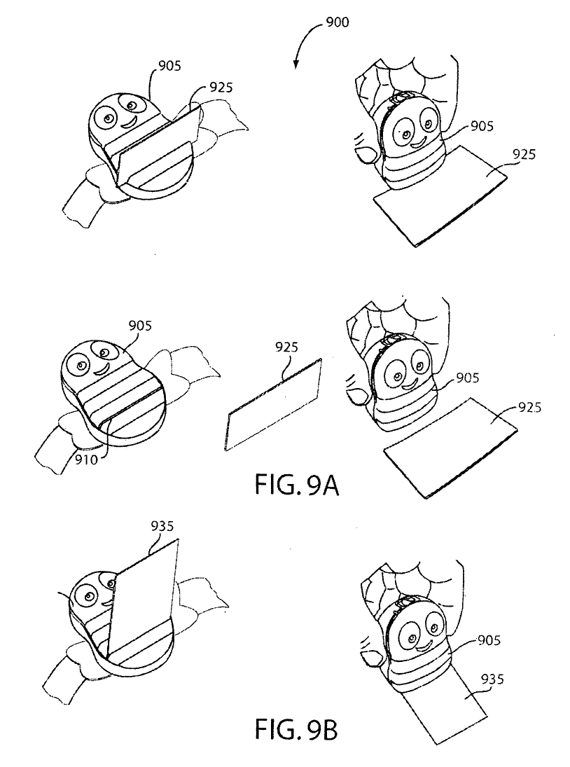

FIG. 9 shows a further embodiment 900 in which a housing 905 is configured with a receiving means 910 for receiving and retaining a focus card 925. As shown in FIG. 9A the focus card 925 can be retained at the housing 905 in a manner that allows a user to view the visual graphics 204. In the example 900, the receiving means 910 is in the form of a slot configured to receive the focus card 925 and retain it in an upright, generally horizontal position. Preferably, a housing retains a focus card in a manner that permits both opposing faces to be visible simultaneously. As shown in FIG. 2, first face 202 includes a margin 206 around its periphery. While the focus card 200 has visual graphics 204 over a substantial portion of the face 202, covering all of the face 202 other than the margin 206, no visual graphics appear in the margin 206. In an example embodiment, the focus card 202 can be retained by a receiving means 910 of a housing 905 so that the margin 206 is covered by the receiving means, but the visual graphics 204 are not obstructed.

In a preferred embodiment, the focus card 925 is secured within the receiving means 910 by a friction fit that holds the focus card 925 securely in place, yet permits easy removal and exchange of the focus card 925. Other means of securing the focus card 925 to the housing 905 may also be employed, including, but not limited to, clasps, fasteners, brackets, hooks, flanges or other means. A focus card may be configured with holes, tabs, notches, or other means to facilitate insertion and retention in receiving means. In the example embodiments of FIGS. 9A, B, the slot 910 is disposed across the housing transverse to a longitudinal axis of the housing, however it is contemplated that a housing can have a slot disposed along its longitudinal axis or at any angle with respect thereto. A focus card can be sized for retention by the receiving means 910. Referring to FIGS. 2 and 9, for a housing such as the example housing 905, the example focus card 925 can have a width w of 9 cm, a length/of 5 cm and a thickness of 0.67 mm.

Referring to FIG. 9 A, the focus card 925 is secured in a generally horizontal orientation within the receiving means 910. However, as shown in FIG. 9B, in a further embodiment the focus card 935 can be retained in a generally vertical orientation. In an example embodiment, the housing 905 can be configured to accommodate a thermal or vibratory analgesia means, or both.

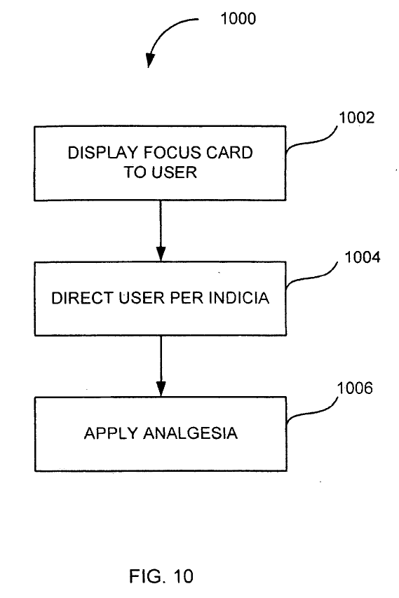

FIG. 10 depicts a flow diagram of an example method 1000. At block 1002 a focus card is displayed to a patient. Referring to FIG. 2, a first face 202 of focus card 200 can be shown to the user 602. The focus card 200 can be shown to the user 602 by the companion individual 603. At block 1004 the accompanying individual 603 can direct the user 602 as per the indicia 214 to focus the user's attention and generate the desired neurological response. In an exemplary embodiment, the individual 603 selects indicia from a group configured for a target population of which user 602 is a member. The patient can provide a verbal or non-verbal response to the accompanying individual 603. The individual 603 can direct the user 602 per another indicia configured for the user's target population. The process can be repeated with a new focus card 620.

As the user 602 focuses his attention on the focus card 200 to respond to the indicia 214 directed to him, a neurological response is induced in which nerve impulses generated in response to focusing his attention on the focus card 200 are conducted along the spinal cord system of the user 602. While the user 602 is focusing his attention on the focus card 200, his neurological response to other stimuli is dampened, as described previously herein. In particular, never impulses associated with the focus card 200 (positive stimulus) interfere with or otherwise impede impulses from other stimuli from crossing the ACG into the cerebral cortex. Because the novel indicia 214 are configured to pertain to the novel visual graphics 204, rather than information or knowledge previously acquired by the user 602, the portion of the brain associated with the user's memory is preferably not activated. It is preferable that the focus card 200 is displayed to the user 602 and the indicia 214 directed to him prior to beginning a medical procedure.

At block 1006, an analgesia means is applied to the patient 602. In an example embodiment, the housing 705 accommodating an analgesia means is positioned on the patient 602 at an area proximate to a medical site. It is preferable that the housing 705 is positioned between the medical site 630 and the nerve plexi (not shown) of the user 602 to increase analgesia effects for the user. It is noted that in an example embodiment, the housing 905 can be positioned on the user 602. The focus card 925 can be received by the receiving means 910, so that the housing 905 retains the focus card 925 rather than a companion holding a focus card. In a preferred embodiment, the housing 905 equipped with the focus card 925 obstructs the user's view of the medical site. In a further embodiment, the focus card 402 is received at the housing 905, and the individual 603 refers to the companion card 408 to direct indicia to the patient 602. At block 1008 an analgesia is applied to the user's body via the analgesia means accommodated within the housing 705. For example, a thermal analgesic means such as a chemical cold pack can be activated so that cold is applied to the patient. As alternative to, or in addition to the thermal analgesic means, a vibratory analgesic means can be activated so that vibrations are applied to the patient. It is preferable that an analgesia means be activated prior to conducting the medical procedure, thereby increasing the analgesic effect for the user 602. Similarly, it is preferred that the analgesic means continue for a short time after conclusion of the medical procedure. In an exemplary example, the method 1000 is practiced prior to the patient undergoing a medical procedure. It is noted that in an example embodiment, that the method 1000 is practiced while a medical procedure is performed on the user 602 at the medical site 630.

The invention described herein provides apparatus, systems, and methods for reducing pain perception in a patient. By focusing a user's attention, the patient's brain response to an afflictive or hurtful stimulus can be dampened. Including an analgesia means, such as thermal and vibratory analgesia, in a system further reduces the patient's pain perception.

A focus card can include visual graphics on a first face, and indicia associated with the visual graphics on a second face. A patient can observe the visual graphics and follow the indicia as directed by a companion or by self-direction. In an exemplary embodiment, the indicia are configured for one or more target populations. For example, a first target population can include children under three years old, while a second target population includes children 3-5 years old. Indicia can be configured for users of a target population to induce the desired neurological response so that a user's perception of pain in response to a negative stimulus is dampened. It is preferable that a person other than the patient, direct the indicia to the user because the social interaction between a user and a companion, as well as the auditory stimulus that further focuses a user's attention on a focus card can further reduce the patient's pain perception. Interaction between the user and a companion is one of several advantages of the present invention over prior art visual distraction techniques.

One or more focus cards can be retained by a retaining means, such as a ring, held by a companion, and shown to the patient one at a time. Alternatively, a housing or other retention device positioned proximate to the user can receive and retain a focus card so that a patient can view the visual graphics of the focus card. A focus card can be configured for coupling to a housing. In a first embodiment, a housing has a receiving means, such as a slot, for receiving a focus card. The housing can be positioned on a patient and retain a focus card in an orientation that allows a patient to view the visual graphics on a first face of the card. In an example embodiment the retained focus card at least partially obstructs the user's view of a medical site on his body. The housing may include one or more analgesia means such as a thermal and/or vibratory analgesia means that can be applied to induce analgesic effects for the patient. In a further embodiment, a housing is positioned in a visually accessible location other than a user's body.

Various embodiments and methods of practicing the invention are disclosed herein. While useful for teaching the invention, they are merely examples and not to be considered limitations on an invention that can be variably practiced. Additional embodiments within the scope of the appended claims will occur to those skilled in the art.

I claim:

1. A focus card, comprising:
a first face having visual graphics covering a substantial portion thereof;
a second face having indicia pertaining to said visual graphics, said indicia configured to focus a user's attention, said indicia configured for a target population identified on said second face; and
wherein said target population identification and said indicia are visually correlated by a common attribute.

2. The focus card of claim 1, wherein said indicia comprise a first group configured for a first target population, and a second group configured for a second target population.

3. The focus card of claim 1, wherein said visual graphics comprise multi-color graphics.

4. The focus card of claim 1, wherein said visual graphics comprise lenticular graphics.

5. The focus card of claim 1, wherein said visual graphics and said indicia are configured to induce a neurological response wherein said user's response to a negative stimulus is reduced.

6. The focus card of claim 5, wherein said visual graphics and said indicia are configured to induce a neurological response wherein said user's sensation of pain is dampened.

7. The focus card of claim 5, configured to evoke a neurological response by said user in which a first group of impulses conducted along said user's spinal cord system in response to said focus card visual graphics and indicia reduce the number of a second group of impulses that pass through a user's substantia gelatinosa to the cerebral cortex, said second group of impulses generated in response to a negative stimulus.

8. The focus card of claim 7, wherein said stimulus is an external stimulus.

9. The focus card of claim 8, wherein said stimulus is a pain-producing stimulus.

10. The focus card of claim 1, wherein said indicia are configured so that said user compliance with said indicia modifies behavior of said user's anterior cingulate gyrus.

11. The focus card of claim 10, wherein said indicia comprise tasks that can be performed by said user without said user recalling from memory previously learned information pertaining to said visual graphics.

12. The focus card of claim 1, configured for retention by a retention means configured to provide an unobstructed view of said visual graphics.

13. The focus card of claim 12, wherein said retention means is configured to provide an unobstructed view of said indicia.

14. A system comprising:
a focus card having first and second opposing faces, said first face having visual graphics on a substantial portion thereof, and said second face having indicia pertaining to said visual graphics configured to focus a user's attention; and
a housing configured to accommodate an analgesic means and apply said analgesic means to a patient's body.

15. The system of claim 14, wherein said analgesic means comprises at least one of a group consisting of a thermal analgesic means and a vibratory analgesic means.

16. The system of claim 14, wherein said indicia are configured for a target population identified on said second face of said focus card.

17. The system of claim 14, comprising a plurality of different said focus cards retained by a retaining means configured for rapid transition between said plurality of focus cards.

18. A method for focusing reducing a user's response to a negative stimulus, comprising:
displaying to a user a first face of a focus card, said first face comprising visual graphics over a substantial portion thereof, said focus card having a second opposing face comprising indicia pertaining to said visual graphics, said indicia configured for a target population identified on said second face, said indicia configured to focus said user's attention on said visual graphics; and
directing said user as indicated by said indicia to focus said user's attention.

19. The method of claim 18, further comprising: in response to said displaying and said directing, evoking a neurological response in said user which dampens said user's response to a negative stimulus.

20. The method of claim 18, further comprising applying to the user's body one of a group consisting of a thermal analgesia and a vibratory analgesia.

21. The method of claim 18, wherein said displaying and said directing are performed while a negative stimulus is applied to said user.

22. The method of claim 18, wherein said negative stimulus is associated with a medical procedure.

23. The method of claim 22, wherein said medical procedure comprises a venipuncture.

24. The method of claim 18, further comprising reducing a patient's perception of pain by modifying behavior of an anterior cingulate gyrus portion of said user's brain.
